(19) United States
(12) Patent Application Publication
(10) Pub. No.: US 2019/0223736 A1
(43) Pub. Date: Jul. 25, 2019

(54) BLOOD PRESSURE TESTING DEVICE AND METHOD

(71) Applicant: BOE TECHNOLOGY GROUP CO., LTD., Beijing (CN)

(72) Inventors: Long WANG, Beijing (CN); Chieh Hsing CHUNG, Beijing (CN); Feng JIANG, Beijing (CN); Yanzhao LI, Beijing (CN)

(73) Assignee: BOE TECHNOLOGY GROUP CO., LTD., Beijing (CN)

(21) Appl. No.: 16/317,653

(22) PCT Filed: May 18, 2018
(86) PCT No.: PCT/CN2018/087506

§ 371 Date: Jan. 14, 2019

(30) Foreign Application Priority Data
May 22, 2017 (CN) 201710368387.X

(51) Int. Cl. A61B 5/021; H01L 41/053; H01L 41/113; H01L 41/193; H01L 41/18; A61B 5/00

(52) U.S. Cl. CPC A61B 5/02108; H01L 41/0533; H01L 41/1132; H01L 41/1138; H01L 41/193; A61B 2560/0214; A61B 5/7278; A61B 5/681; A61B 2562/0247; A61B 2562/164; A61B 2562/046; H01L 41/183

(57) ABSTRACT

A blood pressure measuring device, including a wristband, a control system, and a pressure sensing device, wherein the pressure sensing device is provided on the wristband; the control system is electrically coupled to the pressure sensing device; the pressure sensing device is configured to receive a pressure generated by skin surface and to generated an electrical signal; and the control system is configured to receive the electrical signal and to convert the electrical signal into a pulse wave, and to acquire a blood pressure value based on the pulse wave.

### CROSS-REFERENCE TO RELATED PATENT APPLICATIONS

This application is a U.S. National Stage Application under 35 U.S.C. § 371 of International Patent Application PCT/CN2018/087506 filed May 18, 2018, which claims the benefit of priority under 35 U.S.C. § 119(e) of Chinese Patent Application No.201710368387.X filed on May 22, 2017, both of which are incorporated by reference in their entireties. The International Application was published on 29 Nov. 2018, as International Publication No. WO 2018/214820 A1.

### TECHNICAL FIELD

Embodiments of the present invention relates to a blood pressure measuring device and a blood pressure measuring method.

### BACKGROUND

At present, with the improvement of living conditions and the accelerated pace of life, cardiovascular and cerebrovascular diseases are an important source threatening people's health, and they will take millions of lives every year. Timely and continuous measurement of blood pressure not only helps prevent high blood pressure, but also helps patients with hypertension control blood pressure and maintain their health.

### SUMMARY

At least one embodiment of the present disclosure provided a blood pressure measuring device, comprising a wristband, a control system, and a pressure sensing device, wherein the pressure sensing device is provided on the wristband; the control system is electrically coupled to the pressure sensing device; the pressure sensing device is configured to sense a pressure generated by skin surface and to generated an electrical signal; and the control system is configured to receive the electrical signal and to convert the electrical signal into a pulse wave, and to acquire a blood pressure value based on the pulse wave.

In one embodiment of the present disclosure, the pressure sensing device comprise a pressure sensor substrate and a pressure sensor; the pressure sensor is strip-shaped as a whole, the pressure sensor comprises a first lower electrode layer, a first piezoelectric material layer, and a first upper electrode layer disposed on the pressure sensor substrate in sequence; and the pressure sensor further comprises a first pressure sensor encapsulation protective layer disposed outside.

In one embodiment of the present disclosure, the pressure sensor substrate is a flexible substrate, and the pressure sensor is attached to the wristband or integrated in the wristband.

In one embodiment of the present disclosure, a first insulating protective layer is disposed between the pressure sensor and the pressure sensor substrate, and/or a second insulating protective layer is disposed between the pressure sensor and the first pressure sensor encapsulation protective layer.

In one embodiment of the present disclosure, material for each of the first lower electrode layer is selected as least one of ITO, silver nanowire, silver alloy, graphene, carbon nanotube, carbon nanobud.

In one embodiment of the present disclosure, material for the first upper electrode layer is selected as at least one of ITO, silver nanowire, silver alloy, graphene, carbon nanotube, carbon nanobud.

In one embodiment of the present disclosure, the pressure sensing device comprises a pressure sensor substrate and a pressure sensor, the pressure sensor comprise a plurality of pressure sub-sensors.

In one embodiment of the present disclosure, the plurality of pressure sub-sensors have a shape of strip and are arranged parallel to each other, each of the plurality of pressure sub-sensors comprises a second lower electrode layer, a second piezoelectric material layer and a second upper electrode layer disposed on the pressure sensor substrate in sequence, the second piezoelectric material layer and the second upper electrode layer are each strip-shaped, and the pressure sensor further comprises a second pressure sensor encapsulation protective layer disposed outside.

In one embodiment of the present disclosure, the second lower electrode layers of the plurality of pressure sub-sensors are separated in strips, or formed as an integral structure.

In one embodiment of the present disclosure, a third insulating protective layer is disposed between the second lower electrode layer of each of the plurality of pressure sub-sensors and the pressure sensor substrate, and/or a fourth insulating protective layer is disposed between the second upper electrode layer of each of the plurality of pressure sub-sensors and the second pressure sensor encapsulation protective layer.

In one embodiment of the present disclosure, the plurality of pressure sub-sensors can each have a shape of dot and are arranged in matrix, each of the plurality of pressure sub-sensors comprises a third lower electrode layer, a third piezoelectric material layer, and a third upper electrode layer disposed on the pressure sensor substrate in sequence, each of the third piezoelectric material layer and the third upper electrode layer has a shape of dot, and the pressure sensor further comprises a third pressure sensor encapsulation protective layer is further disposed outside.

In one embodiment of the present disclosure, the third lower electrode layers of the plurality of pressure sensors has a shape of dot and are disposed separately, or, the third lower electrode layers of the plurality of pressure sensors are formed as an integral structure.

In one embodiment of the present disclosure, a fifth insulating protective layer is disposed between the third lower electrode layer of each of the pressure sub-sensors and the pressure sensor substrate, and/or a sixth insulating protective layer is disposed between the third upper electrode layer of each of the pressure sub-sensors and the third pressure sensor encapsulation protective layer.

In one embodiment of the present disclosure, the blood pressure measuring device further comprises a power supply device which comprises a thin film solar cell, the thin film solar cell is disposed on an outwardly facing side of the wristband, and is configured to supply power to the control system and the pressure sensing device.

In one embodiment of the present disclosure, the control system comprises a central processing unit, a pulse data processing unit, and a blood pressure data processing unit; the central processing unit is configured to receive the electrical signal and send the electrical signal to the pulse data processing unit, and is further configured to receive the pulse wave from the pulse data processing unit and send the pulse wave to the blood pressure data processing unit; the pulse data processing unit is configured to receive the electrical signal and convert the electrical signal into the pulse wave, and to transmit the pulse wave to the central processing unit; and the blood pressure data processing unit is configured to receive the pulse wave from the central processing unit, and to calculate a blood pressure value according to the correspondence between the pulse wave and the blood pressure.

In one embodiment of the present disclosure, the pulse data processing unit is further configured to compare the electrical signal with a preset electrical signal threshold range; and to issue prompt information of adjusting wristband if the electrical signal exceeds the preset electrical signal threshold range.

In one embodiment of the present disclosure, the blood pressure data processing unit is further configured to compare the blood pressure value with a preset blood pressure threshold range, and to send prompt information of abnormality to a specific user if the blood pressure value exceeds the preset blood pressure threshold range.

At one embodiment of the present disclosure provides a blood pressure measuring method, comprising: receiving an electrical signal generated by a pressure sensing device due to a pressure exerted thereon by a skin surface; converting the electrical signal into a corresponding pulse wave; and obtaining a blood pressure value according to a correspondence between the pulse wave and a blood pressure.

In one embodiment of the present disclosure, the blood pressure measuring method further comprises comparing the electrical signal with a preset electrical signal threshold range and issuing prompt information of adjusting wristband when the electrical signal exceeds the preset electrical signal threshold range.

In one embodiment of the present disclosure, obtaining the blood pressure value according to the correspondence between the pulse wave and the blood pressure comprising: calculating the blood pressure value by using an approximation and fitting algorithm according to the correspondence between the pulse wave and the blood pressure.

### BRIEF DESCRIPTION OF THE DRAWINGS

In order to clearly illustrate the technical solution of the embodiments of the disclosure, the drawings of the embodiments will be briefly described in the following; it is obvious that the described drawings are only related to some embodiments of the disclosure and thus are not limitative of the disclosure.

FIG. 1 is a schematic structural view of a blood pressure measuring device according to an embodiment of the present disclosure;

FIG. 1a is a schematic structural view of a pressure sensing device in a blood pressure measuring device according to an embodiment of the present disclosure;

FIG. 1b is a schematic structural diagram of a control system in a blood pressure measuring device according to an embodiment of the present disclosure;

FIG. 1c is a schematic diagram illustrating connections of a control system with other units in a blood pressure measuring device according to an embodiment of the present disclosure;

FIG. 2 is a schematic structural view of a blood pressure measuring device according to another embodiment of the present disclosure;

FIG. 2a is a schematic structural view of a pressure sensing device in a blood pressure measuring device according to another embodiment of the present disclosure;

FIG. 2b is a schematic cross-sectional view of the pressure sensing device of FIG. 2a;

FIG. 2c is a schematic structural diagram of a control system in a blood pressure measuring device according to another embodiment of the present disclosure;

FIG. 2d is a schematic diagram illustrating connections of a control system with other units in a blood pressure measuring device according to another embodiment of the present disclosure;

FIG. 3 is a schematic structural view of a blood pressure measuring device according to still another embodiment of the present disclosure;

FIG. 3a is a schematic structural view of a pressure sensing device in a blood pressure measuring device according to still another embodiment of the present disclosure;

FIG. 3b is a schematic structural diagram of a control system in a blood pressure measuring device according to still another embodiment of the present disclosure;

FIG. 3c is a schematic diagram illustrating connections of a control system with other units in a blood pressure measuring device according to still another embodiment of the present disclosure; and

FIG. 4 is a flow diagram of a blood pressure measuring method in accordance with an embodiment of the present disclosure.

### DETAILED DESCRIPTION

In order to make objects, technical details and advantages of the embodiments of the disclosure apparent, the technical solutions of the embodiment will be described in a clearly and fully understandable way in connection with the drawings related to the embodiments of the disclosure. It is obvious that the described embodiments are just a part but not all of the embodiments of the disclosure. Based on the described embodiments herein, those skilled in the art can obtain other embodiment(s), without any inventive work, which should be within the scope of the disclosure.

It should be noted that all expressions using “first” and “second” in the embodiments of the present disclosure are intended to distinguish two entities with the same name that are not the same or non-identical parameters, and “first” and “second” are used for convenience of description, it should not be construed as a limit to the embodiments of the present disclosure, which will not be described in the following specification.

The measurement of blood pressure mainly comprises invasive measurement and non-invasive measurement. Invasive measurement is a direct measurement, the measurement results are more accurate, but not suitable for consumers to self-measure and long-term monitoring. Generally, the invasive measurement can only be carried out in the hospital by a professional doctor; non-invasive measurement, although there will be some errors, but is simple to be carried out. In chronological order, three generations of technology have been developed: the Korotkoff-sound method, the oscillometric method, and the pulse wave method.

The Korotkoff-sound method is mainly to block the blood flow by inflating a cuff, and then slowly deflating the cuff. The tester hears the pulse sound through a stethoscope. The first pulse sound to appear corresponds to the systolic pressure, and with the deflation, the pulse sound disappear, and the last pulse sound corresponds to the diastolic pressure. This method is simple to carry out, but artificial error will be produced due to the operator's seeing and hearing, and is not suitable for continuous measurement.

The principle of the oscillometric method is to use the oscillometric principle of the pulse waveform to estimate the blood pressure based on the relationship between the pulse amplitude and the cuff pressure. The average pressure corresponds to the pulse wave maximum, and the systolic pressures and diastolic pressures are respectively determined according to their respective proportion to the maximum amplitude of the corresponding pulse. Many electronic sphygmomanometers adopt this method with a slightly large error.

The above two blood pressure measurements require a cuff, and a stethoscope or an oscilloscope. There are many devices and the operation is not convenient. It is not suitable for continuous blood pressure measurement and monitoring.

The pulse wave is formed by the spreading of the heart's pulsation along the arterial blood vessels and blood flow to the periphery. With the blood circulation in the blood vessels, the pulse waves at different positions are slightly different. According to the correlation between the pulse wave and the blood flow, the blood pressure can be measured indirectly through the pulse wave. The method adopts multi-point measurement instead of single-point measurement, and the true systolic and diastolic pressure values are calculated by using the approximation and fitting calculation methods on the basis of the internal relationship and variation between the points near the systolic pressure point and diastolic pressure point. Thus, continuous measurement of discrete events can be achieved, more accurate blood pressure values can be measured.

At present, pulse wave blood pressure measuring devices typically require cuff inflation, which is not suitable to monitor blood pressure for long-term.

For patients with cardiovascular and cerebrovascular diseases, high blood pressure and other diseases, it is best to be able to monitor the blood pressure for a long time so as to promptly remind his/her relatives and friends in a critical moment, and to ensure timely treatment. In addition, it has been reported that products adopt the optical method to measure the blood pressure through the pulse wave blood pressure measuring method, however, it is said to be less sensitive.

At least one embodiment of the present disclosure provides a blood pressure measuring device that facilitates users to measure blood pressure and monitor physical condition for a long time.

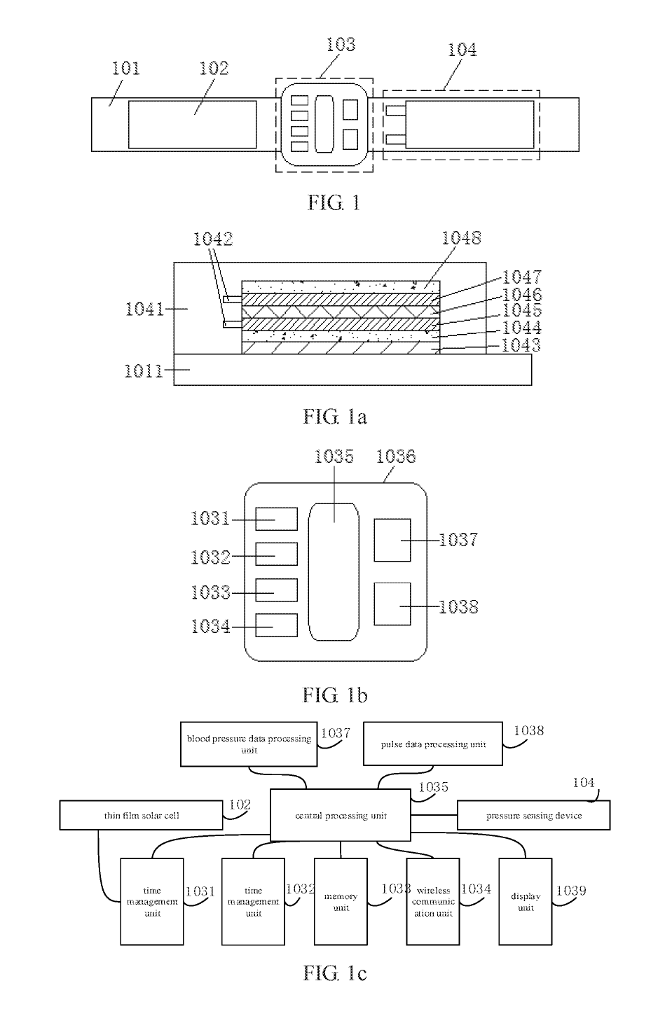

FIG. 1 illustrates a schematic structural view of a blood pressure measuring device according to an embodiment of the present disclosure.

The blood pressure measuring device comprises a wristband 101, a control system 103, and a pressure sensing device 104; the pressure sensing device is provided on the wristband 101, and the control system 103 is electrically coupled to the pressure sensing device 104. Optionally, the wristband 101 has a width ranging from 10 to 30 mm, and can be made of comfortable and flexible silicon, breathable nylon, relatively textured leather or organic elastomer material. Optionally, the blood pressure measuring device further comprises a power supply device, and the power supply device comprises a thin film solar cell 102. As illustrated in FIG. 1, the thin film solar cell 102 is disposed on an outwardly facing side of the wristband 101, and is configured to supply power to the control system 103 and the pressure sensing device 104. Thus, with the solar power supply, the external power supply unit is not required, which is more convenient to use and saves resources. Optionally, the thin film solar cell 102 is integrated or attached to the surface of the wristband 101, and a flexible thin film solar cell, such as an organic solar cell and a dye-sensitized solar cell, can be selected to collect solar energy when the sunlight is present, and charge the blood pressure measuring device, thereby improving standby capacity.

The pressure sensing device 104 is configured to receive pressure generated by a skin surface and generate an electrical signal. Optionally, as illustrated in FIG. 1 and FIG. la, the pressure sensing device 104 comprises a first pressure sensor substrate 1043 and a pressure sensor disposed on the first pressure sensor substrate 1043; the pressure sensor is strip-shaped as a whole, and the pressure sensor comprises a first lower electrode layer 1045, a first piezoelectric material layer 1046, a first upper electrode layer 1047, and a second insulating protective layer 1048 disposed in sequence, and the pressure sensor further comprises a first pressure sensor encapsulation protective layer 1041, and the first lower electrode layer 1045 and the first upper electrode layer 1047 are connected to the control system 103 through the first electrode lead 1042. Optionally, a first insulating protective layer 1044 can be disposed between the first pressure sensor substrate 1043 and the first lower electrode layer 1045. Further, a pressure sensor can be fabricated on a first pressure sensor substrate 1043 which is flexible, and then the pressure sensor as a whole is attached to the wristband base 1011 of the wristband 101 or integrated in the wristband 101.

The first pressure sensor substrate 1043 can be a flexible substrate and can be a flexible polymer material substrate (such as polyimide PI, Polyethylene terephthalate PET, or other flexible polymer material), and the pressure sensor is fabricated on the flexible substrate, and the first insulating protective layer 1044 can be made of a polymer material, silicon nitride or silicon oxide; the first lower electrode layer 1045 can configured as a common electrode layer, and can be made of a transparent conductive film and has a thickness ranging from 0.5 μm to 10 μm. Material for the first lower electrode layer 1045 can be selected from: ITO, silver nanowire, silver alloy, graphene, carbon nanotube, carbon nanobud and etc. The first piezoelectric material layer 1046 can convert a pressure signal into an electrical signal, and has a thickness ranging from 10 μm to 200 μm. Piezoelectric materials that can be used are: piezoelectric polymer (polyvinylidene fluoride, polyvinyl fluoride, polyvinyl chloride, isobutylene, methyl methacrylate, a vinyl benzoate or the like and a copolymer thereof), and a composite material of a piezoelectric ceramic and a polymer, and the like. The first upper electrode layer 1047 is configured as a signal transmission layer, and similar to the first lower electrode layer 1045, the first upper electrode layer 1047 can be made of transparent conductive film and has a thickness ranging from 0.5 μm to 10 μm. Material for the first upper electrode layer 1047 can be selected from ITO, silver nanowire, silver alloy, graphene, carbon nanotube, carbon nanobud, and etc. The second insulating protective layer 1048 is prevented from contacting the first upper electrode layer 1047 and the first pressure sensor encapsulation protective layer 1041. The first pressure sensor encapsulation protective layer 1041 can be made of the same material as the wristband substrate 1011. Optionally, the overall thickness of the pressure sensor along with the first pressure sensor encapsulation protective layer 1041 is controlled between 0.5 and 2 mm

The control system 103 is configured to receive the electrical signal and convert the electrical signal into a corresponding pulse wave, and to calculate a blood pressure value according to the correspondence between the pulse wave and the blood pressure. And optionally, the blood pressure measuring device further comprises a display unit 1039 (referring to FIG. 1c) configured to display the blood pressure value.

Optionally, as illustrated in FIG. 1b and FIG. 1c, the control system 103 is a central control and data processing area of the blood pressure measuring device, and has a size of less than 30*30 mm The control system 103 comprises a central processing unit 1035, a pulse data processing unit 1038, and a blood pressure data processing unit 1037.

The central processing unit 1035 is configured to receive the electrical signal and send the electrical signal to the pulse data processing unit 1038, and receive the pulse wave and send the pulse wave to the blood pressure data processing unit 1037. Optionally, the central processing unit 1035 is further configured to receive a blood pressure value from the blood pressure data processing unit 1037 and transmits the blood pressure value to the display unit 1039 for display. The central processing unit 1035 can be implemented by a processing chip such as an MCU or a DSP.

The pulse data processing unit 1038 is configured to receive the electrical signal and convert the electrical signal into a pulse wave, and to transmit the pulse wave to the central processing unit 1035. Optionally, the pulse data processing unit 1038 is further configured to compare the electrical signal with a preset electrical signal threshold range; and issue prompt information of adjusting wristband if the electrical signal exceeds the preset electrical signal threshold range. In this way, by comparing the electrical signal with a preset electrical signal threshold range, when the electrical signal exceeds the preset electrical signal threshold range, it is determined that the current wristband should be too tight or too loose, thereby enabling adjusting the wristband in time so as to measure the blood pressure value accurately.

The blood pressure data processing unit 1037 is configured to receive the pulse wave, and to calculate a blood pressure value according to the correspondence between the pulse wave and the blood pressure. Optionally, The blood pressure data processing unit 1037 is further configured to return the blood pressure value to the central processing 1035. Optionally, the blood pressure data processing unit 1037 calculates the blood pressure value by using an approximation and fitting algorithm according to the correspondence between the pulse wave and the blood pressure. Thus, by using this algorithm for calculating the blood pressure value, a relatively accurate blood pressure value can be obtained. Optionally, the blood pressure data processing unit 1037 is further configured to compare the blood pressure value with a preset blood pressure threshold range, and to send prompt information of abnormality to a specific user if the blood pressure value exceeds the preset blood pressure threshold range. In this way, by comparing the blood pressure value with the preset blood pressure threshold range, when the blood pressure value exceeds the preset blood pressure threshold range, it is determined that the current blood pressure value is abnormal, thereby issuing prompt information of abnormality to the specific user, so that timely attention is paid. For patients with underlying hypertension, when monitoring data changes abnormally, the person under test and the scheduled person can be remaindered by a short message of paying attention to the physical condition of the tester in time, thereby playing a role of reminding and preventing, that is, when the blood pressure is obviously fluctuating, the wearer is remaindered so that the wearer can go to the hospital for examination and early treatment in time.

Optionally, as illustrated in FIG. 1b and FIG. 1c, the control system 103 further comprises a time management unit 1032, a memory unit 1033, a wireless communication unit 1034, and a power management unit 1031.

The time management unit 1032 is configured to send a blood pressure test trigger command to the central processing unit 1035 at predetermined time intervals, thereby enabling continuous measuring and monitoring of blood pressure values. In addition, the time management unit 1032 can be further configured to periodically issue a medication reminder triggering command to the central processing unit 1035, so that auxiliary disease management and reminding timing medication for hypertensive patients can be achieved. The time management unit 1032 can be implemented as a timing circuit connected to the central processing unit 1035, or can be implemented as a software program embedded in the central processing unit 1035, or can be implemented as a combination of a timing circuit in the central processing unit 1035 and a software program embedded in the central processing unit 1035.

The memory unit 1033 is configured to store measurement data; such that the blood pressure measuring device has a memory function for convenient daily use, and is suitable for long-term monitoring and blood pressure management. The measurement data can comprise blood pressure values and pulse waves. The memory unit 1033 can also be configured to store prompt information, such as prompt information for reminding medication or prompt information sent to the person under test or a specific person. The memory unit 1033 can also be configured to store contact information of the person under test and contact information of a specific person, and the like. The memory unit can be implemented as a memory circuit. The memory unit can employ a commercially available memory unit. The memory unit can also be a cloud storage unit or the like, and the measurement data can be transmitted to the cloud storage unit through a wireless communication unit, and can be downloaded from the cloud storage unit to the blood pressure measuring device through a wireless communication unit.

The wireless communication unit 1034 is configured to implement a wireless communication connection with an external terminal; the measurement data can also be transmitted by the wireless communication unit 1034 to a mobile terminal such as an external mobile phone or a tablet computer, thereby facilitating management of measurement data. The wireless communication unit 1034 can employ Bluetooth technology, infrared communication technology, near field communication technology, cellular communication technology, and the like.

The power management unit 1031 is configured to perform unified management control on the power source. Optionally, the power management unit 1031 can further comprise a battery, and the blood pressure measuring device is powered by the battery. The power management unit 1031 can further comprise a thin film solar cell 102 disposed on the wristband 101 as a supplement to the battery. When the battery power is in low power, the thin film solar cell 102 can be used as a supplement to the battery, and the blood pressure measuring device can be powered, or the battery can be charged by a wired or wireless charging method.

As illustrated in FIG. 1c, optionally, the control system 103 further comprises a display unit 1039. The display unit 1039 is configured to display the blood pressure value and the prompt information and the display unit 1039 is not necessary for the device. And optionally, to ensure low power consumption, a LED display with low resolution or an EPD display, or a reflective LCD display can be used. Optionally, the control system 103 may not comprise a display unit, and the control system 103 can be wirelessly connected to an external terminal through the wireless communication unit 1034, and the measurement data can be displayed and managed through the external terminal (such as a mobile phone).

The blood pressure measuring working mode of the blood pressure measuring device can be: first, the blood pressure measuring device is worn on the wrist, and a portion of the wristband where the pressure sensor is located covers the pulse beating region. The beating the pulse at the wrist is sensed by a pressure sensor, the first piezoelectric material layer 1046 is slightly deformed after being subjected to a force, and an electrical signal corresponding to the deformation is output; the electrical signal is sent via the central processing unit 1035 to the pulse data processing unit 1038, the pulse data processing unit 1038 can convert the electrical signal into a pulse wave, and then send the pulse wave to the central processing unit 1035, and then the central processing unit 1035 sends the pulse wave to the blood pressure data processing unit 1037, and the blood pressure data processing unit 1037 calculates the blood pressure value by the approximation and fitting algorithm based on the correspondence between the pulse wave and the blood pressure. In addition, the pulse data processing unit 1038 can also calculate and output a pulse figure according to the change of the electrical signal. The blood pressure measuring device can be connected to a mobile device as needed so as to view and manage the measurement data. With this portable device, the blood pressure measurement operation process can be greatly simplified, and continuous measuring can be realized. It can also help to analyze the physical condition through pulse figure information, and then the blood pressure measuring device will become a personal health assistant for the wearer.

In the blood pressure measuring device according to the embodiment of the present disclosure, the pressure generated by the skin surface is received by the pressure sensing device and an electrical signal is generated accordingly, and then the electrical signal is converted into a pulse wave, and the blood pressure value is calculated according to the correspondence between the pulse wave and the blood pressure. Because of high sensitivity of the pressure sensor, a more accurate blood pressure value can be obtained, which is convenient for consumers to measure blood pressure and monitor physical condition for a long time. In addition, the blood pressure measuring device can analyze the health condition of the person under test through software or hardware by means of the pulse beating information corresponding to the acquired pulse wave, and initially has the function of taking pulse in the Chinese medicine. Compared to an inflatable sphygmomanometer, the blood pressure measuring device is convenient for daily use and is suitable for blood pressure monitoring and management for long-term. For patients with hypertension, blood pressure can be measured in time so as to remind patients with hypertension of taking medicine.

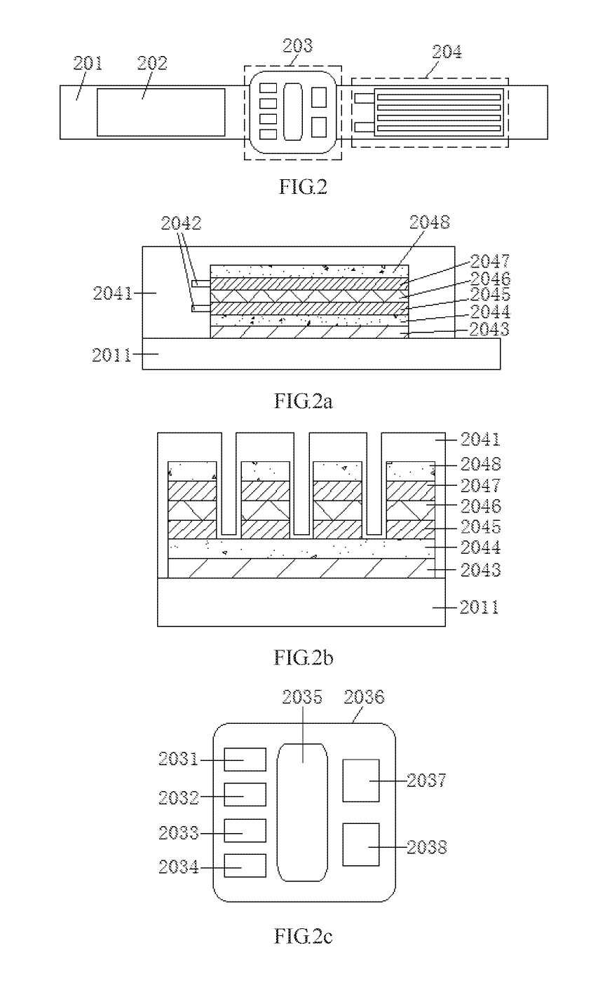

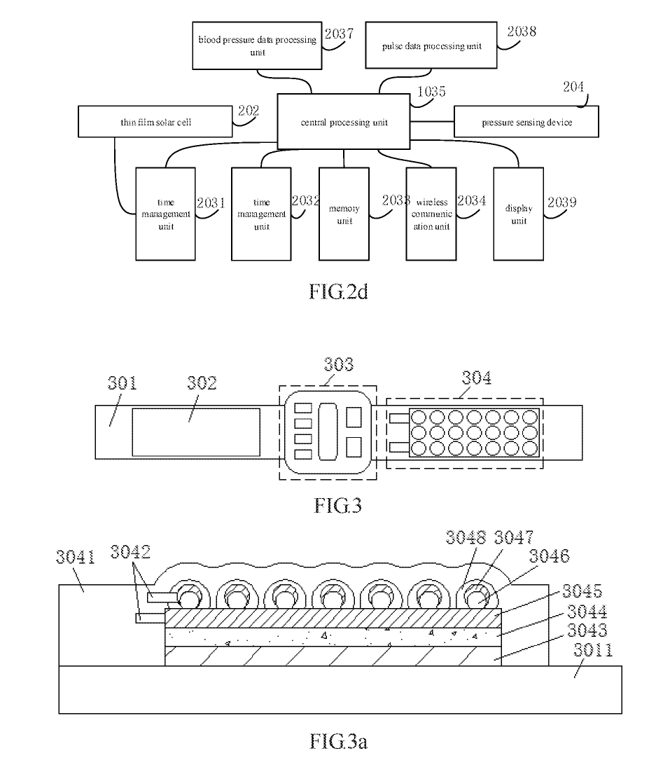

FIG. 2 illustrate a schematic structural view of a blood pressure measuring device according to another embodiment of the present disclosure.

The blood pressure measuring device comprises a wristband 201, a control system 203, and a pressure sensing device 204; the pressure sensing device 204 is provided on the wristband 201, and the control system 203 is electrically coupled to the pressure sensing device 204. Optionally, the wristband 201 has a width ranging from 10 to 30 mm, and can be made of comfortable and flexible silicon, breathable nylon, relatively textured leather or organic elastomer material. Optionally, the blood pressure measuring device further comprises a power supply device, and the power supply device comprises a thin film solar cell 202. The thin film solar cell 202 is disposed on an outwardly facing side of the wristband 201, and is configured to supply power to the control system 203 and the pressure sensing device 204. Thus, with the solar power supply, the external power supply unit is not required, which is more convenient to use and saves resources. Optionally, the thin film solar cell 202 is integrated or attached to the surface of the wristband 201, and a flexible thin film solar cell, such as an organic solar cell and a dye-sensitized solar cell, can be selected to collect solar energy when the sunlight is present, and charge the blood pressure measuring device, thereby improving standby capacity.

The pressure sensing device 204 is configured to receive a pressure generated by a skin surface and generate an electrical signal; optionally, as illustrated in FIG. 2, FIG. 2a and FIG. 2b, the pressure sensing device 204 comprises a pressure sensor; the pressure sensor comprises a second pressure sensor substrate 2043 and a plurality of pressure sub-sensors disposed on the second pressure sensor substrate 2043.

Wherein, as illustrated in FIG. 2, FIG. 2a and FIG. 2b, the plurality of pressure sub-sensors can be a plurality of first pressure sub-sensors which have a shape of strip and are arranged in parallel on the second pressure sensor substrate 2043, each of the plurality of pressure sub-sensors comprises a second lower electrode layer 2045, a second piezoelectric material layer 2046, a second upper electrode layer 2047, and a fourth insulating protective layer 2048, provided in sequence. The second piezoelectric material layer 2046, the second upper electrode layer 2047 and the fourth insulating protective layer 2048 are each strip-shaped; the pressure sensor further comprises a second pressure sensor encapsulation protective layer 2041 disposed outside. The second lower electrode layer 2045 and the second upper electrode layer 2047 are connected to the control system 203 through the second electrode lead 2042. The second lower electrode layer 2045 of each of the plurality of pressure sub-sensors can be in the form of a strip, and be disposed corresponding to the piezoelectric material layer 2046, as illustrated in FIG. 2b, or the second lower electrode layers 2045 of the plurality of pressure sub-sensors can be formed as an integral structure formed on the second pressure sensor substrate 2043 (not illustrated). Optionally, a third insulating protective layer 2044 is disposed between the plurality of pressure sub-sensors and the second pressure sensor substrate 2043. Further, the pressure sensor can be fabricated on a flexible second pressure sensor substrate 2043, and then the pressure sensor as a whole is adhered to the wristband base 2011 of the wristband 201 or integrated in the wristband 201. In this way, by using a plurality of strip-shaped first pressure sub-sensors, a plurality of electrical signals can be collected to monitor richer pulse beating information, thereby obtaining a more accurate blood pressure value.

The second pressure sensor substrate 2043 can be a flexible substrate and can be a flexible polymer material substrate (such as polyimide PI, Polyethylene terephthalate PET, or other flexible polymer material), and the pressure sensor is fabricated on the flexible substrate. The third insulating protective layer 2044 can be made of a polymer material, silicon nitride or silicon oxide; the second lower electrode layer 2045 can configured as a common electrode layer, and can be made of a transparent conductive film and has a thickness ranging from 0.5 μm to 10 μm. Material for the second lower electrode layer 2045 can be selected from: ITO, silver nanowire, silver alloy, graphene, carbon nanotube, carbon nanobud and etc. The second piezoelectric material layer 2046 can convert a pressure signal into an electrical signal, and has a thickness ranging from 10 μm to 200 μm. Piezoelectric materials that can be used are: piezoelectric polymer (polyvinylidene fluoride, polyvinyl fluoride, polyvinyl chloride, isobutylene, methyl methacrylate, a vinyl benzoate or the like and a copolymer thereof), and a composite material of a piezoelectric ceramic and a polymer, and the like. The second upper electrode layer 2047 is configured as a signal transmission layer, and similar to the second lower electrode layer 2045, the second upper electrode layer 1047 can be made of transparent conductive film and has a thickness ranging from 0.5 μm to 10 μm. Material for the second upper electrode layer 2047 can be selected from ITO, silver nanowire, silver alloy, graphene, carbon nanotube, carbon nanobud, and etc. The fourth insulating protective layer 2048 is prevented from contacting the second upper electrode layer 2047 and the second pressure sensor encapsulation protective layer 2041. The second pressure sensor encapsulation protective layer 2041 can be made of the same material as the wristband substrate 2011. Optionally, the overall thickness of the pressure sensor along with the second pressure sensor encapsulation protective layer 2041 is controlled between 0.5 and 2 mm.

The control system 203 is configured to receive the electrical signal and convert the electrical signal into a corresponding pulse wave, and to calculate a blood pressure value according to the correspondence between the pulse wave and the blood pressure. And optionally, the blood pressure measuring device further comprises a display unit 2039 (referring to FIG. 2d) configured to display the blood pressure value.

Optionally, as illustrated in FIG. 2c and FIG. 2d, the control system 203 is a central control and data processing area of the blood pressure measuring device, and has a size of less than 30*30 mm The control system 203 comprises a central processing unit 2035, a pulse data processing unit 2038, and blood pressure data processing unit 2037.

The central processing unit 2035 is configured to receive the electrical signal from the pressure sensing device 204 and send the electrical signal to the pulse data processing unit 2038, and the central processing unit 2035 is further configured to receive the pulse wave from the pulse data processing unit 2038 and send the pulse wave to the blood pressure data processing unit 2037. Optionally, the central processing unit 2035 is further configured to receive a blood pressure value from the blood pressure data processing unit 2037 and transmits the blood pressure value to the display unit 2039 for display. The central processing unit 2035 can be implemented by a processing chip such as an MCU or a DSP.

The pulse data processing unit 2038 is configured to receive the electrical signal and convert the electrical signal into a pulse wave, and to transmit the pulse wave to the central processing unit 2035. Optionally, the pulse data processing unit 2038 is further configured to compare the electrical signal with a preset electrical signal threshold range; and issue prompt information of adjusting wristband if the electrical signal exceeds the preset electrical signal threshold range. In this way, by comparing the electrical signal with a preset electrical signal threshold range, when the electrical signal exceeds the preset electrical signal threshold range, it is determined that the current wristband should be too tight or too loose, thereby enabling adjusting the wristband in time so as to measure the blood pressure value accurately.

The blood pressure data processing unit 2037 is configured to receive the pulse wave, and to calculate a blood pressure value according to the correspondence between the pulse wave and the blood pressure. Optionally, the blood pressure data processing unit 2037 is further configured to return the blood pressure value to the central processing 2035. Optionally, the blood pressure data processing unit 2037 calculates the blood pressure value by using an approximation and fitting algorithm according to the correspondence between the pulse wave and the blood pressure. Thus, by using this algorithm for calculating the blood pressure value, a relatively accurate blood pressure value can be obtained. Optionally, the blood pressure data processing unit 2037 is further configured to compare the blood pressure value with a preset blood pressure threshold range, and to send prompt information of abnormality to a specific user if the blood pressure value exceeds the preset blood pressure threshold range. In this way, by comparing the blood pressure value with the preset blood pressure threshold range, when the blood pressure value exceeds the preset blood pressure threshold range, it is determined that the current blood pressure value is abnormal, thereby issuing prompt information of abnormality to the specific user, so that timely attention is paid. For patients with underlying hypertension, when monitoring data changes abnormally, the person under test and the scheduled person (such as a doctor, a nurse, or his/her family) can be remaindered by a short message of paying attention to the physical condition of the tester in time, thereby playing a role of reminding and preventing, that is, when the blood pressure is obviously fluctuating, the wearer is remaindered so that the wearer can go to the hospital for examination and early treatment in time.

Optionally, as illustrated in FIG. 2c and FIG. 2d, the control system 203 further comprises a time management unit 2032, a memory unit 2033, a wireless communication unit 2034, and a power management unit 2031.

The time management unit 2032 is configured to send a blood pressure measure trigger command to the central processing unit 2035 at predetermined time intervals, thereby enabling continuous measuring and monitoring of blood pressure values. In addition, the time management unit 2032 can be further configured to periodically issue a medication reminder triggering command to the central processing unit 2035, so that auxiliary disease management and reminding timing medication for hypertensive patients can be achieved. The time management unit 2032 can be implemented as a timing circuit connected to the central processing unit 2035, or can be implemented as a software program embedded in the central processing unit 2035, or can be implemented as a combination of a timing circuit in the central processing unit 2035 and a software program embedded in the central processing unit 1035.

The memory unit 2033 is configured to store measurement data; such that the blood pressure measuring device has a memory function for convenient daily use, and is suitable for long-term monitoring and blood pressure management. The measurement data can comprise blood pressure values and pulse waves. The memory unit 2033 can also be configured to store prompt information, such as prompt information for reminding medication or prompt information sent to the person under test or a specific person. The memory unit 2033 can also be configured to store contact information of the person under test and contact information of a specific person, and the like. The memory unit can be implemented as a memory circuit. The memory unit can employ a commercially available memory unit. The memory unit can also be a cloud storage unit or the like, and the measurement data can be transmitted to the cloud storage unit through a wireless communication unit, and can be downloaded from the cloud storage unit to the blood pressure measuring device through a wireless communication unit.

The wireless communication unit 2034 is configured to implement a wireless communication connection with an external terminal; the measurement data can also be transmitted by the wireless communication unit 2034 to a mobile terminal such as an external mobile phone or a tablet computer, thereby facilitating management of measurement data. The wireless communication unit 2034 can employ Bluetooth technology, infrared communication technology, near field communication technology, cellular communication technology, and the like.

The power management unit 2031 is configured to perform unified management control on the power source. Optionally, the power management unit 2031 can further comprise a battery, and the blood pressure measuring device is powered by the battery. The power management unit 2031 can further comprise a thin film solar cell 202 disposed on the wristband 201 as a supplement to the battery. When the battery power is in low power, the thin film solar cell 202 can be used as a supplement to the battery, and the blood pressure measuring device can be powered, or the battery can be charged by a wired or wireless charging method.

Optionally, the control system 203 further comprises a display unit 2039. The display unit 2039 is configured to display the blood pressure value and the prompt information and the display unit 2039 is not necessary for the device. And optionally, to ensure low power consumption, a LED display with low resolution or an EPD display, or a reflective LCD display can be used. Optionally, the control system 203 may not comprise a display unit, and the control system 203 can be wirelessly connected to an external terminal through the wireless communication unit 2034, and the measurement data can be displayed and managed through the external terminal (such as a mobile phone).

The blood pressure measuring working mode of the blood pressure measuring device can be: first, the blood pressure measuring device is worn on the wrist, and a portion of the wristband where the pressure sensor is located covers the pulse beating region. The beating the pulse at the wrist is sensed by a pressure sensor, the second piezoelectric material layer 2046 is slightly deformed after being subjected to a force, and an electrical signal corresponding to the deformation is output; the electrical signal is sent via the central processing unit 2035 to the pulse data processing unit 2038, the pulse data processing unit 2038 can convert the electrical signal into a pulse wave, and then send the pulse wave to the central processing unit 2035, and then the central processing unit 2035 sends the pulse wave to the blood pressure data processing unit 2037, and the blood pressure data processing unit 2037 calculates the blood pressure value, for example, by the approximation and fitting algorithm based on the correspondence between the pulse wave and the blood pressure. In addition, the pulse data processing unit 2038 can also calculate and output a pulse figure according to the change of the electrical signal. The blood pressure measuring device can be connected to a mobile device as needed so as to view and manage the measurement data. With this portable device, the blood pressure measurement operation process can be greatly simplified, and continuous measuring can be realized. It can also help to analyze the physical condition through pulse figure information, and then the blood pressure measuring device will become a personal health assistant for the wearer.

In the blood pressure measuring device according to the embodiment of the present disclosure, the pressure generated by the skin surface is received by the pressure sensing device and an electrical signal is generated accordingly, and then the electrical signal is converted into a pulse wave, and the blood pressure value is calculated according to the correspondence between the pulse wave and the blood pressure. Because of high sensitivity of the pressure sensor, a more accurate blood pressure value can be obtained, which is convenient for consumers to measure blood pressure and monitor physical condition for a long time. In addition, the blood pressure measuring device can analyze the health condition of the person under test through software or hardware by means of the pulse beating information corresponding to the acquired pulse wave, and initially has the function of taking pulse in the Chinese medicine. Compared to an inflatable sphygmomanometer, the blood pressure measuring device is convenient for daily use and is suitable for blood pressure monitoring and management for long-term. For patients with hypertension, blood pressure can be measured in time so as to remind patients with hypertension of taking medicine.

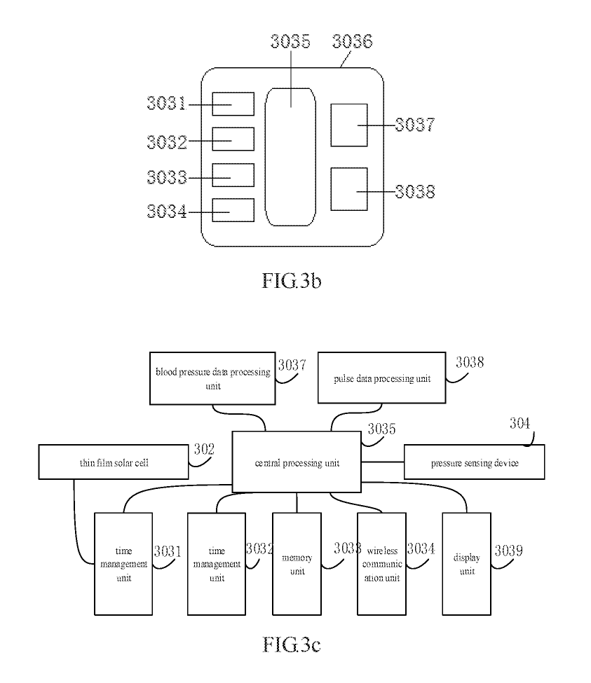

FIG. 3 illustrate a schematic structural view of a blood pressure measuring device according to another embodiment of the present disclosure.

The blood pressure measuring device comprises a wristband 301, a control system 303, and a pressure sensing device 304; the pressure sensing device 304 is provided on the wristband 301, and the control system 303 is electrically coupled to the pressure sensing device 204. Optionally, the wristband 301 has a width ranging from 10 to 30 mm, and can be made of comfortable and flexible silicon, breathable nylon, relatively textured leather or organic elastomer material. Optionally, the blood pressure measuring device further comprises a power supply device, and the power supply device comprises a thin film solar cell 302. The thin film solar cell 302 is disposed on an outwardly facing side of the wristband 301, and is configured to supply power to the control system 303 and the pressure sensing device 304. Thus, with the solar power supply, the external power supply unit is not required, which is more convenient to use and saves resources. Optionally, the thin film solar cell 302 is integrated or attached to the surface of the wristband 301, and a flexible thin film solar cell, such as an organic solar cell and a dye-sensitized solar cell, can be selected to collect solar energy when the sunlight is present, and charge the blood pressure measuring device, thereby improving standby capacity.

The pressure sensing device 304 is configured to receive a pressure generated by a skin surface and generate an electrical signal; optionally, as illustrated in FIG. 3 and FIG. 3a, the pressure sensing device 304 comprises a third pressure sensor substrate 3043 and a pressure sensor disposed on the third pressure sensor substrate 2043, and the pressure sensor comprises a plurality of second pressure sub-sensors.

As illustrated in FIG. 3, and FIG. 3a, the plurality of second pressure sub-sensors each can have a shape of dot and are arranged in matrix. Each of the plurality of second pressure sub-sensors comprises a third lower electrode layer 3045, a third piezoelectric material layer 3046, a third upper electrode layer 3047, and a sixth insulating protective layer 3048, provided in sequence. The third piezoelectric material layer 3046, and the third upper electrode layer 3047 are each in form of dot. The third lower electrode layer 3045 can be in form of dot corresponding to the third piezoelectric material layer 3046, or the third lower electrode layer of the plurality of second pressure sub-sensors are formed into an integral structure, and disposed on the third pressure sensor substrate 3043. The sixth insulating protective layer 3048 of each of the plurality of second pressure sub-sensors can be shaped into dot, and cover the third upper electrode layer. Or, the sixth insulating layer of each of the plurality of second pressure sub-sensors are formed into an integral structure and cover the plurality of second pressure sub-sensors entirely. The pressure sensor further comprises a third pressure sensor encapsulation protective layer 3041 disposed outside. Optionally, a fifth insulating protective layer 3044 is disposed between the pressure sensor and the third pressure sensor substrate 3043. The third lower electrode layer 3045 and the third upper electrode layer 3047 are connected to the control system 303 through the third electrode lead 3042 (due to the limit of size of the figure, only the third electrode lead 3042 of the left-most second pressure sub-sensor is illustrated in FIG. 3a, and the third electrode lead 3042 of other second pressure sub-sensors are omitted). Further, the pressure sensor can be fabricated on the third flexible pressure sensor substrate 3043, and then the pressure sensor as a whole is adhered to the wristband base 3011 of the wristband 301 or integrated in the wristband 301. In this way, by using a plurality of second pressure sub-sensors in form of dot, better contact with the wrist can be achieved, multiple electrical signals can be collected, and more abundant pulse beating information can be monitored, thereby obtaining a more accurate blood pressure value. Optionally, the third lower electrode layer 3045 of the pressure sensor is formed as a single piece of electrodes (as illustrated in FIG. 3a), and the third upper electrode layer 3047 is designed according to an electrode pattern, and is formed into discrete electrodes by sputtering or vapor deposition. Alternatively, the third lower electrode layer 3045 of each of the second pressure sub-sensors is separately provided (not illustrated).

The third pressure sensor substrate 3043 can be a flexible substrate and can be a flexible polymer material substrate (such as polyimide PI, Polyethylene terephthalate PET, or other flexible polymer material), and the pressure sensor is fabricated on the flexible substrate. The fifth insulating protective layer 3044 can be made of a polymer material, silicon nitride or silicon oxide; the third lower electrode layer 3045 can configured as a common electrode layer, and can be made of a transparent conductive film and has a thickness ranging from 0.5 μm to 10 μm. Material for the third lower electrode layer 3045 can be selected from: ITO, silver nanowire, silver alloy, graphene, carbon nanotube, carbon nanobud and etc. The third piezoelectric material layer 3046 can convert a pressure signal into an electrical signal, and has a thickness ranging from 10 μm to 200 μm. Piezoelectric materials that can be used are: piezoelectric polymer (polyvinylidene fluoride, polyvinyl fluoride, polyvinyl chloride, isobutylene, methyl methacrylate, a vinyl benzoate or the like and a copolymer thereof), and a composite material of a piezoelectric ceramic and a polymer, and the like. The third upper electrode layer 3047 is configured as a signal transmission layer, and similar to the third lower electrode layer 3045, the third upper electrode layer 1047 can be made of transparent conductive film and has a thickness ranging from 0.5 μm to 10 μm. Material for the third upper electrode layer 3047 can be selected from ITO, silver nanowire, silver alloy, graphene, carbon nanotube, carbon nanobud, and etc. The sixth insulating protective layer 3048 is disposed to be prevented from contacting the third upper electrode layer 3047 and the third pressure sensor encapsulation protective layer 3041. The third pressure sensor encapsulation protective layer 3041 can be made of the same material as the wristband substrate 3011. Optionally, the overall thickness of the pressure sensor along with the third pressure sensor encapsulation protective layer 3041 is controlled between 0.5 and 2 mm.

The control system 303 is configured to receive the electrical signal and convert the electrical signal into a corresponding pulse wave, and to calculate a blood pressure value according to the correspondence between the pulse wave and the blood pressure. And optionally, the blood pressure measuring device further comprises a display unit 3039 (referring to FIG. 3c) configured to display the blood pressure value.

Optionally, as illustrated in FIG. 3b and FIG. 3c, the control system 303 is a central control and data processing area of the blood pressure measuring device, and has a size of less than 30*30 mm. The control system 303 comprises a central processing unit 3035, a pulse data processing unit 3038, and blood pressure data processing unit 3037.

The central processing unit 3035 is configured to receive the electrical signal and send the electrical signal to the pulse data processing unit 3038, and the central processing unit 3035 is further configured to receive a pulse wave from the pulse data processing unit 3038 and send the pulse wave to the blood pressure data processing unit 3037. Optionally, the central processing unit 3035 is further configured to transmit the blood pressure value to the display unit 3039 for display. The central processing unit 3035 can be implemented by a processing chip such as an MCU or a DSP.

The pulse data processing unit 2038 is configured to receive the electrical signal from the central processing unit and convert the electrical signal into a pulse wave, and to transmit the pulse wave back to the central processing unit 3035. Optionally, the pulse data processing unit 3038 is further configured to compare the electrical signal with a preset electrical signal threshold range; and issue prompt information of adjusting wristband if the electrical signal exceeds the preset electrical signal threshold range. In this way, by comparing the electrical signal with a preset electrical signal threshold range, when the electrical signal exceeds the preset electrical signal threshold range, it is determined that the current wristband should be too tight or too loose, thereby enabling adjusting the wristband in time so as to measure the blood pressure value accurately.

The blood pressure data processing unit 3037 is configured to receive the pulse wave, and to calculate a blood pressure value according to the correspondence between the pulse wave and the blood pressure. Optionally, the blood pressure data processing unit 3037 is further configured to return the blood pressure value to the central processing unit 3035. Optionally, the blood pressure data processing unit 3037 calculates the blood pressure value by using an approximation and fitting algorithm according to the correspondence between the pulse wave and the blood pressure. Thus, by using this algorithm for calculating the blood pressure value, a relatively accurate blood pressure value can be obtained. Optionally, the blood pressure data processing unit 3037 is further configured to compare the blood pressure value with a preset blood pressure threshold range, and to send prompt information of abnormality to a specific user if the blood pressure value exceeds the preset blood pressure threshold range. In this way, by comparing the blood pressure value with the preset blood pressure threshold range, when the blood pressure value exceeds the preset blood pressure threshold range, it is determined that the current blood pressure value is abnormal, thereby issuing prompt information of abnormality to the specific user, so that timely attention is paid. For patients with underlying hypertension, when monitoring data changes abnormally, the person under test and the scheduled person (such as a doctor, a nurse, or his/her family) can be remaindered by a short message of paying attention to the physical condition of the tester in time, thereby playing a role of reminding and preventing, that is, when the blood pressure is obviously fluctuating, the wearer is remaindered so that the wearer can go to the hospital for examination and early treatment in time.

Optionally, as illustrated in FIG. 3b and FIG. 3c, the control system 303 further comprises a time management unit 3032, a memory unit 3033, a wireless communication unit 3034, and a power management unit 3031.

The time management unit 3032 is configured to send a blood pressure measure trigger command to the central processing unit 3035 at predetermined time intervals, thereby enabling continuous measuring and monitoring of blood pressure values. In addition, the time management unit 2032 can be further configured to periodically issue a medication reminder triggering command to the central processing unit 3035, so that auxiliary disease management and reminding timing medication for hypertensive patients can be achieved. The time management unit 3032 can be implemented as a timing circuit connected to the central processing unit 3035, or can be implemented as a software program embedded in the central processing unit 3035, or can be implemented as a combination of a timing circuit in the central processing unit 3035 and a software program embedded in the central processing unit 3035.

The memory unit 3033 is configured to store measurement data; such that the blood pressure measuring device has a memory function for convenient daily use, and is suitable for long-term monitoring and blood pressure management. The measurement data can comprise the electrical signal, the blood pressure values and the pulse waves. The memory unit 3033 can also be configured to store prompt information, such as prompt information for reminding medication or prompt information sent to the person under test or a specific person. The memory unit 3033 can also be configured to store contact information of the person under test and contact information of a specific person, and the like. The memory unit can be implemented as a memory circuit. The memory unit can employ a commercially available memory unit. The memory unit 3033 can also be a cloud storage unit or the like, and the measurement data can be transmitted to the cloud storage unit through a wireless communication unit, and can be downloaded from the cloud storage unit to the blood pressure measuring device through a wireless communication unit.

The wireless communication unit 3034 is configured to implement a wireless communication connection with an external terminal; the measurement data can also be transmitted by the wireless communication unit 3034 to a mobile terminal such as an external mobile phone or a tablet computer, thereby facilitating management of measurement data. The wireless communication unit 3034 can employ Bluetooth technology, infrared communication technology, near field communication technology, cellular communication technology, and the like.

The power management unit 3031 is configured to perform unified management control on the power source. Optionally, the power management unit 3031 can further comprise a battery, and the blood pressure measuring device is powered by the battery. The power management unit 3031 can further comprise a thin film solar cell 302 disposed on the wristband 301 as a supplement to the battery. When the battery power is in low power, the thin film solar cell 302 can be used as a supplement to the battery, and the blood pressure measuring device can be powered by the thin film solar cell 302, or the battery can be charged by a wired or wireless charging method.

Optionally, the control system 303 further comprises a display unit 3039. The display unit 3039 is configured to display the blood pressure value and the prompt information and the display unit 3039 is not necessary for the device. And optionally, to ensure low power consumption, a LED display with low resolution or an EPD display, or a reflective LCD display can be used. Optionally, the control system 303 may not comprise a display unit, and the control system 303 can be wirelessly connected to an external terminal through the wireless communication unit 3034, and the measurement data can be displayed and managed through the external terminal (such as a mobile phone).

The blood pressure measuring working mode of the blood pressure measuring device can be: first, the blood pressure measuring device is worn on the wrist, and a portion of the wristband where the pressure sensor is located covers the pulse beating region. The beating the pulse at the wrist is sensed by a pressure sensor, the second piezoelectric material layer 3046 is slightly deformed after being subjected to a force, and an electrical signal corresponding to the deformation is output; the electrical signal is sent via the central processing unit 3035 to the pulse data processing unit 3038, the pulse data processing unit 3038 can convert the electrical signal into a pulse wave, and then send the pulse wave to the central processing unit 3035, and then the central processing unit 3035 sends the pulse wave to the blood pressure data processing unit 3037, and the blood pressure data processing unit 3037 calculates a blood pressure value based on the correspondence between the pulse wave and the blood pressure. For example, the blood pressure value can be calculated by the approximation and fitting algorithm based on the correspondence between the pulse wave and the blood pressure. In addition, the pulse data processing unit 3038 can also calculate and output a pulse figure according to the change of the electrical signal. The blood pressure measuring device can be connected to a mobile device as needed so as to transmit the measurement data to the mobile device to display and/or manage the measurement data, facilitating viewing and managing the measurement. With this portable device, the blood pressure measurement operation process can be greatly simplified, and continuous measuring can be realized. It can also help to analyze the physical condition through pulse figure information, and then the blood pressure measuring device will become a personal health assistant for the wearer.

In the blood pressure measuring device according to the embodiment of the present disclosure, the pressure generated by the skin surface is received by the pressure sensing device and an electrical signal is generated accordingly, and then the electrical signal is converted into a pulse wave, and the blood pressure value is calculated according to the correspondence between the pulse wave and the blood pressure. Because of high sensitivity of the pressure sensor, a more accurate blood pressure value can be obtained, which is convenient for consumers to measure blood pressure and monitor physical condition for a long time. In addition, the blood pressure measuring device can analyze the health condition of the person under test through software or hardware by means of the pulse beating information corresponding to the acquired pulse wave, and initially has the function of taking pulse in the Chinese medicine. Compared to an inflatable sphygmomanometer, the blood pressure measuring device is convenient for daily use and is suitable for blood pressure monitoring and management for long-term. For patients with hypertension, blood pressure can be measured in time so as to remind patients with hypertension of taking medicine.

It should be particularly noted that, in the above embodiments, the blood pressure measuring device is implemented by using a first pressure sub-sensor in a shape of strip and a second pressure sub-sensor in a shape of dot, but the two embodiments can be combined so as to form one embodiment, in which both the first pressure sub-sensor in the form of strip and the second pressure sub-sensor in the form of dot are comprised in the pressure sensing device.

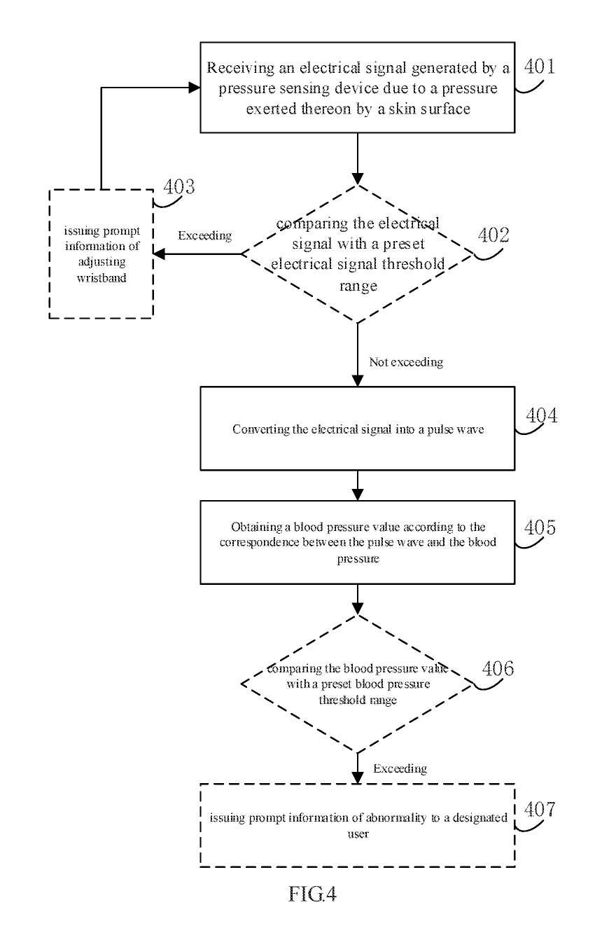

At least one embodiment of the present disclosure provides a blood pressure measuring method that facilitates measurement of blood pressure and monitoring of physical condition of a consumer for long term. FIG. 4 illustrates a flow diagram of a blood pressure measuring method in accordance with one embodiment of the present disclosure.

The blood pressure measuring method comprising the following steps:

Step 401: Receiving an electrical signal generated by a pressure sensing device due to a pressure exerted thereon by a skin surface; optionally, at predetermined time intervals, a time management unit issuing an instruction to a central processing unit to measure blood pressure and/or pulse, and receiving an electrical signal generated by the pressure sensing device due to the pressure exerted thereon by the skin surface.

Because the pressure sensor is disposed in an area where the pulse beating information is easy to capture, in order to ensure the accuracy of the measurement data, it is judged whether the wristband wearing state is appropriate or not according to the pressure data reflected by the electrical signal measured by the pressure sensor before formal measuring, if not, it is required to adjust degree of tightness and position of the wristband. Generally, it needs to tighten or loosen the wristband a little. Therefore, optionally, the measuring method further comprises: Step 402: comparing the electrical signal with a preset electrical signal threshold range; wherein the preset electrical signal threshold range is set and adjusted according to actual operating parameters, which is not limited herein.

Step 403: If the electrical signal exceeds the preset electrical signal threshold range, issuing prompt information of adjusting wristband, and return to step 401; thus, by comparing the electrical signal with a preset electrical signal threshold range, when the electrical signal exceeds the preset electrical signal threshold range, it is determined that the wristband should be too tight or too loose currently, so that the wristband can be adjusted in time to measure a relatively accurate blood pressure value.

Step 404: Converting the electrical signal into a pulse wave if the electrical signal does not exceed the preset electrical signal threshold range.

Step 405: Obtaining a blood pressure value according to the correspondence between the pulse wave and the blood pressure. For example, using an approximation and fitting algorithm, the blood pressure value is calculated according to the correspondence between the pulse wave and the blood pressure; optionally, measurement data (comprising electrical signals, pulse waves, blood pressure values, etc.) can be stored in a memory unit. In addition, the measurement data can also be transmitted to mobile terminals such as mobile phones and tablet computers through a wireless communication unit to facilitate data management. The measurement data can also be stored in the cloud to facilitate data recovery and use. For example, the measurement data can be uploaded to the cloud through a wireless communication unit, and the measurement data can be downloaded from the cloud to the blood pressure measuring device or a mobile phone, a tablet computer, etc. through the wireless communication unit when needed.

Optionally, the blood pressure measuring method can further comprise step 406: comparing the blood pressure value with a preset blood pressure threshold range; wherein the preset blood pressure threshold range can be set adjusted according to actual user requirement, which is not limited herein.

Step 407: If the blood pressure value exceeds the preset blood pressure threshold range, issuing prompt information of abnormality to a designated user. Optionally, when the monitoring data changes abnormally, the prompt information of abnormality can be sent to the person under test or scheduled persons by short message through the wireless communication unit so as to pay attention to the physical condition of the tester in time; thus, by comparing the blood pressure value with the preset blood pressure threshold range, when the blood pressure value exceeds the preset blood pressure threshold range, it is determined that the current blood pressure value is abnormal, so that abnormal prompt message of abnormality is sent to the designated user to reminder the designated user to pay attention to the abnormality in time.

Optionally, for the hypertensive patient, the blood pressure measuring method can further comprise periodically issuing a reminder medication triggering command, thereby achieving an effect of assisting the disease management and reminding of taking medicine in time.

In addition, optionally, the prompt information of adjusting wristband and the prompt information of abnormality are further displayed on a display unit disposed on the blood pressure measuring device, so that an external mobile terminal is not required to display the prompt information. Of course, the two manners can be combined to provide a double reminder, in order to prevent users from missing information. Alternatively, the prompt information of abnormality can be sent to a specific person, for example, a relative of the person under test or a medical staff, and the specific person is remaindered of paying attention to the physical condition of the person under test and to provide necessary assistance and protection to the person under test.

In the blood pressure measuring method according to the embodiment of the present disclosure, the pressure generated by the skin surface is received by the pressure sensing device and an electrical signal is generated accordingly, and then the electrical signal is converted into a pulse wave, and the blood pressure value is calculated according to the correspondence between the pulse wave and the blood pressure. Because of high sensitivity of the pressure sensor, a more accurate blood pressure value can be obtained, which is convenient for consumers to measure blood pressure and monitor physical condition for a long time. In addition, the blood pressure measuring method can analyze the health condition of the person under test through software by means of the pulse beating information corresponding to the acquired pulse wave, and initially has the function of taking pulse in the Chinese medicine. Compared to an inflatable sphygmomanometer, the blood pressure measuring method is convenient for daily use and is suitable for blood pressure monitoring and management for long-term. For patients with hypertension, blood pressure can be measured in time so as to remind patients with hypertension of taking medicine.

The foregoing are merely exemplary embodiments of the disclosure, but are not used to limit the protection scope of the disclosure. The protection scope of the disclosure shall be defined by the attached claims.

In the claims:

1. A blood pressure measuring device, comprising a wristband, a control system, and a pressure sensing device, wherein
the pressure sensing device is provided on the wristband;
the control system is electrically coupled to the pressure sensing device;
the pressure sensing device is configured to receive a pressure generated by skin surface and to generated an electrical signal; and
the control system is configured to receive the electrical signal and to convert the electrical signal into a pulse wave, and to acquire a blood pressure value based on the pulse wave.

2. The blood pressure measuring device according to claim 1, wherein the pressure sensing device comprise a pressure sensor substrate and a pressure sensor;
the pressure sensor is strip-shaped as a whole, the pressure sensor comprises a first lower electrode layer, a first piezoelectric material layer, and a first upper electrode layer disposed on the pressure sensor substrate in sequence; and
the pressure sensor further comprises a first pressure sensor encapsulation protective layer disposed outside.

3. The blood pressure measuring device according to claim 2, wherein the pressure sensor substrate is a flexible substrate, and the pressure sensor is attached to the wristband or integrated in the wristband.

4. The blood pressure measuring device according to claim 2, wherein a first insulating protective layer is disposed between the pressure sensor and the pressure sensor substrate, and/or a second insulating protective layer is disposed between the pressure sensor and the first pressure sensor encapsulation protective layer.

5. The blood pressure measuring device according to claim 2, wherein material for each of the first lower electrode layer and the first upper electrode layer is selected as least one of ITO, silver nanowire, silver alloy, graphene, carbon nanotube, carbon nanobud.

6. The blood pressure measuring device according to claim 2, wherein material for the first upper electrode layer is selected as at least one of ITO, silver nanowire, silver alloy, graphene, carbon nanotube, carbon nanobud.

7. The blood pressure measuring device according to claim 1, wherein the pressure sensing device comprises a pressure sensor substrate and a pressure sensor, the pressure sensor comprise a plurality of pressure sub-sensors.

8. The blood pressure measuring device according to claim 7, wherein the plurality of pressure sub-sensors have a shape of strip and are arranged parallel to each other, each of the plurality of pressure sub-sensors comprises a second lower electrode layer, a second piezoelectric material layer and a second upper electrode layer disposed on the pressure sensor substrate in sequence, the second piezoelectric material layer and the second upper electrode layer are each strip-shaped, and the pressure sensor further comprises a second pressure sensor encapsulation protective layer disposed outside.

9. The blood pressure measuring device according to claim 8, wherein the second lower electrode layers of the plurality of pressure sub-sensors are separated in strips, or formed as an integral structure.

10. The blood pressure measuring device according to claim 8, wherein a third insulating protective layer is disposed between the second lower electrode layer of each of the plurality of pressure sub-sensors and the pressure sensor substrate, and/or a fourth insulating protective layer is disposed between the second upper electrode layer of each of the plurality of pressure sub-sensors and the second pressure sensor encapsulation protective layer.

11. The blood pressure measuring device according to claim 7, wherein the plurality of pressure sub-sensors can each have a shape of dot and are arranged in matrix, each of the plurality of pressure sub-sensors comprises a third lower electrode layer, a third piezoelectric material layer, and a third upper electrode layer disposed on the pressure sensor substrate in sequence, each of the third piezoelectric material layer and the third upper electrode layer has a shape of dot, and the pressure sensor further comprises a third pressure sensor encapsulation protective layer is further disposed outside.

12. The blood pressure measuring device according to claim 11, wherein the third lower electrode layers of the plurality of pressure sensors has a shape of dot and are disposed separately, or, the third lower electrode layers of the plurality of pressure sensors are formed as an integral structure.

13. The blood pressure measuring device according to claim 12, wherein a fifth insulating protective layer is disposed between the third lower electrode layer of each of the pressure sub-sensors and the pressure sensor substrate, and/or a sixth insulating protective layer is disposed between the third upper electrode layer of each of the pressure sub-sensors and the third pressure sensor encapsulation protective layer.

14. The blood pressure measuring device according to claim 1, further comprising a power supply device which comprises a thin film solar cell, the thin film solar cell is disposed on an outwardly facing side of the wristband, and is configured to supply power to the control system and the pressure sensing device.

15. The blood pressure measuring device according to claim 1, wherein the control system comprises a central processing unit, a pulse data processing unit, and a blood pressure data processing unit;
the central processing unit is configured to receive the electrical signal and send the electrical signal to the pulse data processing unit, and is further configured to receive the pulse wave from the pulse data processing unit and send the pulse wave to the blood pressure data processing unit;
the pulse data processing unit is configured to receive the electrical signal and convert the electrical signal into the pulse wave, and to transmit the pulse wave to the central processing unit; and
the blood pressure data processing unit is configured to receive the pulse wave from the central processing unit, and to calculate a blood pressure value according to the correspondence between the pulse wave and the blood pressure.

16. The blood pressure measuring device according to claim 15, wherein the pulse data processing unit is further configured to compare the electrical signal with a preset electrical signal threshold range; and to issue prompt information of adjusting wristband if the electrical signal exceeds the preset electrical signal threshold range.

17. The blood pressure measuring device according to claim 15, wherein the blood pressure data processing unit is further configured to compare the blood pressure value with a preset blood pressure threshold range, and to send prompt information of abnormality to a specific user if the blood pressure value exceeds the preset blood pressure threshold range.

18. A blood pressure measuring method, comprising:
receiving an electrical signal generated by a pressure sensing device due to a pressure exerted thereon by a skin surface;
converting the electrical signal into a corresponding pulse wave; and
obtaining a blood pressure value according to a correspondence between the pulse wave and a blood pressure.

19. The blood pressure measuring method according to claim 18, further comprising:
comparing the electrical signal with a preset electrical signal threshold range; and
issuing prompt information of adjusting wristband when the electrical signal exceeds the preset electrical signal threshold range.

20. The blood pressure measuring method according to claim 18, wherein obtaining the blood pressure value according to the correspondence between the pulse wave and the blood pressure comprising:
calculating the blood pressure value by using an approximation and fitting algorithm according to the correspondence between the pulse wave and the blood pressure.
